(19) United States
(12) Patent Application Publication
(10) Pub. No.: US 2025/0292903 A1
(43) Pub. Date: Sep. 18, 2025

(54) METHODS FOR ESTIMATING AND SERVING WAKE WINDOW PREDICTIONS BASED ON SLEEP DATA

(71) Applicant: Huckleberry Labs, Inc., Irvine, CA (US)

(72) Inventors: Glen Ferguson, Great Neck, NY (US); Tina Kim, San Jose, CA (US); Amber Lore, Smithtown, NY (US); Vila Navazo, Sabadell (ES); Juan Pablo Gavagnin, Ciudad Autonoma De Buenos Aires (AR); Daniel Huang, Chestnut Hill, MA (US); Seng Oon Toh, Riverside, CA (US); Jessica Toh, Riverside, CA (US)

(21) Appl. No.: 18/608,002

(22) Filed: Mar. 18, 2024

(51) Int. Cl. G16H 50/20; G06N 5/02

(52) U.S. Cl. CPC G16H 50/20; G06N 5/02

(57) ABSTRACT

According to certain aspects of the present disclosure, systems and methods are disclosed for tracking and predicting the optimal wake windows of individual infants at scale and thus, the optimal sleep times of a user on a screen customized to each child prioritizing the use of highly personalized values based on machine learning to augment existing expert opinions of wake windows by age that will be automatically adaptive to the changes in child sleep and to generate predictions personalized for each child.

### TECHNICAL FIELD

The present disclosure relates generally to estimating a subject infant's optimal sleep times and, in particular, to systems and methods for estimating and customizing precise predictions of an infant's actual sleep patterns based on statistical modeling that incorporates data analysis from the most recent infant sleep data, expert determined values, and data collected from a large statistically significant base of users.

### BACKGROUND

Difficulty in helping infants to sleep for longer periods of time and in a consistent manner over time is one of the most challenging situations for parents and caregivers. When babies do not sleep for sufficient periods of time and/or in a scheduled manner it reduces the parents' quality of life and disturbs the parents' amount of sleep causing anxiety, stress and mental health issues so addressing infant sleep can lead to increased indexes of happiness. The level of sleep deprivation can be severe in certain instances resulting in emotional and psychological damage to the child, parents and family. In many states maternity leave is 12 weeks or less meaning that many families will have two working parents. Thus, the lack of infant sleep can also result in knock-on effects for the family. These impacts will include challenges at performing technical work, challenges in maintaining healthy habits, irritability, depression and negative impacts on maternal attitude. Sleep is unique to each individual and has a distribution of differences over children. Children have day night cycles that can be different, the amount of time between sleep and wakefulness is different, and the number of naps over the same ages can be different. These differences indicate that personalization is important when attempting to improve baby sleep quality with the intention of improving the family dynamic.

Different methods of helping children to sleep range the gamut from night lights (for those sensitive to darkness), apparel/sleepsacks (for comfort for the baby), blackout curtains (for those sensitive to light), humidifiers (for those affected by dry air), white noise machines/background music (to soothe the baby), sleep training (forcible attempts to regulate the baby's schedules), sleep stories (to cause the baby to fall asleep), and other user apps including the “napper” app that tells parents when to put down their child to sleep/apps that suggest sleep schedules based on predetermined “average” sleep and wake times defined by certain experts. These methods are inadequate in that they typically only help “soothe” a parent's attempt to help the baby sleep without any consideration for optimal or “sweetspot” time-frames where it is more likely for the baby to fall asleep based on their own circadian rhythms, bodily cycles, and their individual environments. Separate from medical challenges that hurt sleep, the state of the art is bespoke plans for individual children. Known solutions use expert-determined wake window values (i.e. the expected average times when an infant would be expected to be conscious before falling asleep) based on the child's age and the number of naps the child is currently taking. This system is equivalent to a rule-based system, but lacks personalization based on the actual trends and patterns seen with the child. Further, these solutions are based on general models that may or may not apply to any individual infant and can only serve, at best, as guidelines without accounting for the actual sleep/wake windows of the infant in question. What is needed is a solution that offers improved accuracy by offering customized recommendations of sleep from observed patterns logged while keeping within the guidelines of accepted wake windows by not deviating by more than a significant percentage of the default wake windows. The rule-based system is brittle with respect to changes in sleep patterns, local differences, and changes in the statistical distribution of the data. Rule-based systems require frequent updates and patches to work at the same level of accuracy.

No existing solutions are known that use machine learning or artificial intelligence (AI). Currently, professional sleep consultants use rules of thumb and child specific qualities to arrive at wake windows that make sense for the specific child. This method is the state of the art solution to determining wake windows for children.

The foregoing general description and the following detailed description are exemplary and explanatory only and are not restrictive of the disclosure. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section. This system described here uses predictive modeling of infant sleep based on actual sleep patterns for children with expert determined wake windows to predict the wake windows based on a recent log of sleep times over recent days of sleep. The values are screened for data quality and missing values are filled with values determined by experts. The model was built based on nearly 2 million users with 450 million wake windows recorded of their infant to build the model, train it and input parameters from the last relevant period of days-when the infant slept and for how long, when the infant woke up and for how long, each successive sleep time period and wake window period and the utilization of fixed wake windows based on experience and applying machine learning to train the AI model which can still work with incomplete data in the most recent wake period and the process of identifying what features are relevant including the average amount of wake time along with multiple data features as defined below.

Data optimization is done on a per child basis on a large scale instead of doing anything across a data set so that the model completes its analysis at the level of each individual child. All child data is individualized and is used to train at a global scale involving all of the wake window data from various users and translated into 50 million days of data across the user base. The system then predicts data for each individual child that is itemized at a child's level looking at multiple data features including: day start, previous night end, total previous wake windows for the most recent defined days (for purposes of this embodiment uses last 5 days), all previous naps of the infant in that recent period, and all of the sleep and wake windows for each day with their previous values labeled wake window 1 and so on.

Limits are used to ensure that the wake window predictions for the infants never fall significantly outside of the expert determined values for the wake windows. The system can have values that differ significantly from the real values when the child rapidly oscillates between high and low sleep needs. The statistical model will make predictions that are based on a predetermined optimal recent period which in this embodiment is the last five days. At values where there are large changes in child sleep the expert determined median values will be more accurate. When there are values that are higher and lower than average or far outside the median the model will reflect back to the accepted sleep consultant value. Thus, these values are favored in cases when there is a large difference from the expert values. The wake window as noted above is the time period when a child wakes and when they are ready to go to sleep the next time.

The age range for the audience of the product ranges from 2 months old going forward as there are no circadian rhythms below 2 months old infants (i.e. the natural ability to recognize when day or night). The model lasts until the infant turns 1 year old. The embodiment incorporates 20 distinct machine learning models that include a prediction between different feature areas. That is, for every wake window or nap number, there is a corresponding value that is each independent for each wake window for each baby. A block of code is trained on all data wherein the last 5 days are averaged and other previous values are utilized. These wake windows are predefined by ages and number of naps and the set of static tables developed by sleep consultants based on data they were seeing in the app.

### SUMMARY

According to certain aspects of the present disclosure, systems and methods are disclosed for tracking and predicting the wake windows of individual infants at scale and thus, the optimal sleep times of a user on a screen customized to each child prioritizing the use of highly personalized values based on machine learning to augment existing expert opinions of wake windows by age. Thus, the system is less biased with respect to change as it will be automatically adaptive to the changes in child sleep. The values used to make predictions are personalized for each child. No child is at the exact mean or median value. They have changes that are not captured in these values of the wake windows determined by industry standards. These values are used for safeguards or as a means of imputing missing data.

In some embodiments, a method comprises an end-to-end system starting from user inputted data to deliver customized predictions for when a young child will next sleep comprising: systems for storing data; feature engineering; model training; data drift detection; model retraining, and model serving of customized wake window predictions for young children; receiving inputs from a user taken by a app input device having a screen; receiving nap times and wake times; inputting the series of nap times and wake times into a data storage medium that is pre trained to analyze various aspects of the data, thereby extracting a plurality of features from the series of inputted data to train and analyze a machine learning model to generate internal prediction of optimal sleep window predictions based on the value of recent history.

In some embodiments, the machine learning model is pre trained by: receiving a individualized dataset of wake and sleep windows; receiving a ground truth wake and sleep windows determined by user input; parsing each individualized features of into a series of dataset wake and sleep windows; inputting the extracted features and the ground truth to the machine learning model, LightGBM, using industry standard techniques.

In some embodiments, the system receives a individualized dataset of wake and sleep windows inputting such data into the data storage medium and and a custom screening system designed by sleep experts is used to determine whether the individualized inputted dataset is within acceptable ranges and parsing and selecting the inputted data to determine appropriate values and features for predicted sleep windows.

In some embodiments, the system receives a individualized dataset of wake and sleep windows inputting such data into the data storage medium and a custom screening system designed by sleep experts is used to determine whether the individualized inputted dataset is within acceptable ranges and if not in acceptable ranges parsing and selecting expert determined wake window standards to replace output generated from the inputted data to determine appropriate values and features for predicted sleep windows.

In some embodiments, the system is able to analyze various features of sleep and wake windows based on timing, trends, durations, and wake times.

In some embodiments, the method further comprises the determination of at least one pattern between extracted features of sleep and wake windows; and associating each extracted feature with a labeled predicted sleep window based on each feature set.

In some embodiments, patterns are determined across the series of imputed datasets and normalized sleep and wake windows.

In some embodiments, certain inputted data features are given higher relevance with respect to inputted data features that are outside of accepted values.

In some embodiments, the method further comprises a rule to use the predictive models with current sleep values to present wake window predictions if the user has not been online for up to and including a certain minimum period of days.

In some embodiments, the method further comprises where if the time the user has not logged in is greater than the optimized time period for inputted values, the value for the wake window determined by sleep experts is used as the prediction.

### BRIEF DESCRIPTION OF THE DRAWINGS

The accompanying drawing, which is incorporated into and constitutes a part of this specification, illustrates various exemplary embodiments and together with the description, serve to explain the principles of the disclosed embodiments.

FIG. 1 is a flowchart illustrating an exemplary method for determining customized predictions of sleep windows for infants, according to techniques disclosed herein.

FIG. 2 is an exemplary computing node.

### DETAILED DESCRIPTION

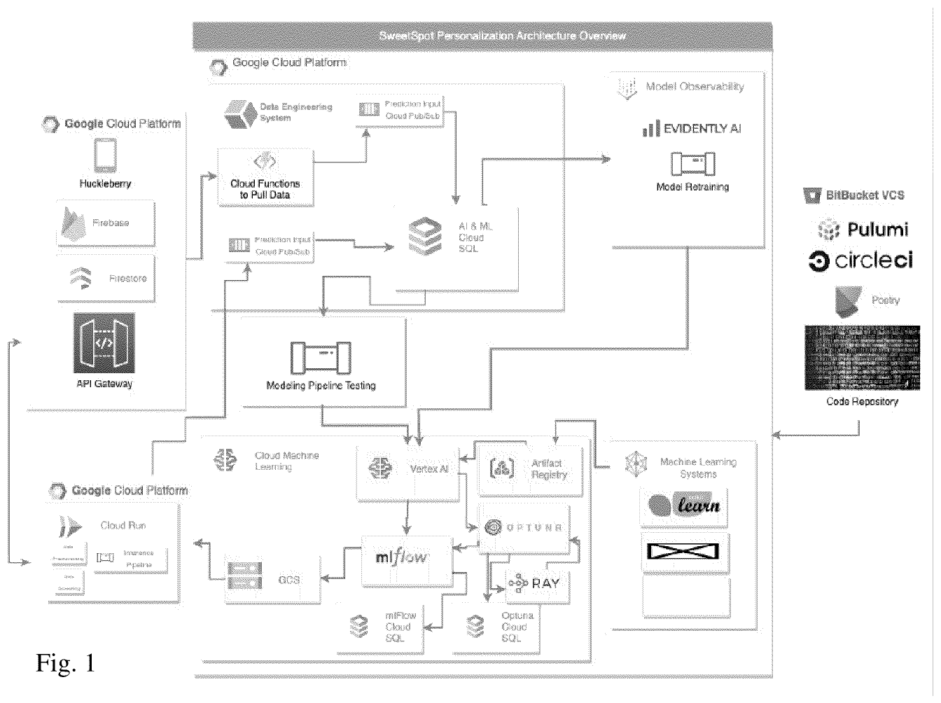

FIG. 1 illustrates the disclosure of an end-to-end system starting from user inputted data to deliver customized predictions for when a baby will next sleep. Elements included are systems for storing data, feature engineering, model training, data drift detection, model retraining, and model serving. The system accepts input from an external web application for the times of sleep starts for the baby and sleep ends for the entered values and then uses a fault tolerant system to load the data into an transactional database. Feature engineering at the level of the individual is done in the transactional database. The features for sleep times include start, sleep times from the previous night, the previous day's sleep, and the previous naps. These values are then fed into a system to perform individualized predictions for the number of naps and the wake window for the baby. A wake window is the time a baby is awake between naps after which optimal sleep times for the baby are served to parents to optimize sleep. Default wake windows and sleep windows generated by expert sleep consultants are used to fill in missing values not provided by parents or caregivers. The invention necessitates statistically significant values involving millions of days of wake windows were to train the initial models. The models are trained using for this embodiment, Vertex AI, a Google Cloud Platform (GCP) product based on Kube Flow to train the models at scale. All experiments are tracked and high-level model parameters optimized. These data points are stored for this embodiment in Cloud SQL databases. The models for this invention embodiment are moved into a docker container run using GCP Cloud Run and container registry.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

FIG. 1 illustrates how the system is queried and using current data, predictions are made and passed to the application and to an additional fault tolerant system for storage. A custom system is used to observe when the values are drifting from those used in training which triggers retraining for the system.

FIG. 1 shows, in addition, a custom system was developed for model serving that includes limits of differences from the Sleep Consultant values for different age groupings. The advantages of the system are fault tolerance, that is hands off for all processes, and can easily be triggered by any authenticated process with minimal data to maximize resources at a minimal cost.

More specifically, the application is developed inside the Google Cloud Platform (GCP) used by the company to store data and serve information to the users. In this embodiment, the system is divided into the data engineering infrastructure and the data science infrastructure. In the data engineering infrastructure Cloud Functions are used to receive notifications of updates which are pushed to a Cloud SQL database using a Cloud Pub/Sub for a queue that is resilient to failures. Once in the database, these data fields are transformed into sets of wake windows, naps, and ages. These transformed values are used to train a machine learning model to predict the wake windows using the mean of the last five values of the variables, previous wake windows, and the previous night's sleep. These values are used in machine learning models to predict the actual wake windows for the child. The training is done using GCP products and the values for training and hyperparameter optimization meaning that the model has parameters trained on data. The models are added to a Docker container in a GCP container register in a Cloud Run used to serve predictions. The values used to make the predictions are sent from the front-end of the application and are screened and pre-processed prior to being used for inference. If the data fails the screening process, they are not pre-processed and inferences are not done. Inferences are requested via an API Gateway in GCP as needed. The values served from the model are done with the data treated in the exact same fashion as the training data. Predictions are screened to ensure they are not significantly outside the default sleep consultant values. If so, the default expert values are used. Thus, the system is able to blend between the two types of prediction even if there is insufficient data which can switch over to the machine learning model.

The system predicts based on the relative interaction of all of these factors together in which it is looking for large decision trees and aggregating to understand the best possible fit using hyper parameter tuning. The system is doing the data-prediction individualization at scale and then the data is trained to develop personalized values at scale. There are two different processing concurrently proceeding-taking data and transforming it into customized predictive wake window times and taking previous values from feature engineering to manipulate sleep and wake times for predicting wake window values, nap numbers and transform it into the features including age, nap #, wake windows, day starts, night total, previous night start, etc.

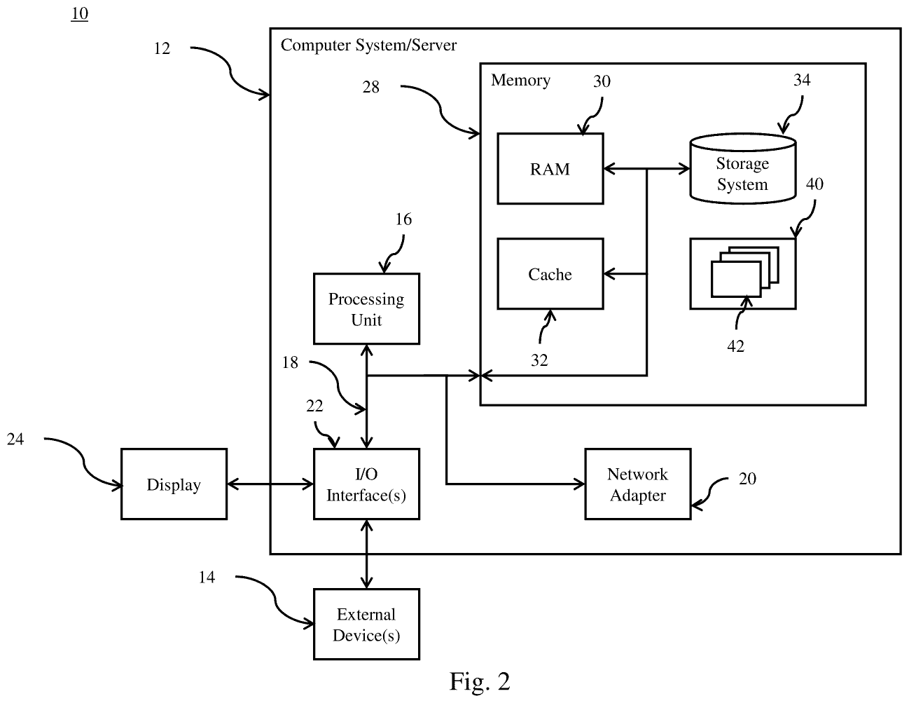

Referring now to FIG. 2, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 2, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed:

1. A method, comprising:
an end-to-end system starting from user inputted data to deliver customized predictions for when a young child will next sleep comprising:
systems for storing data;
feature engineering;
model training;
data drift detection;
model retraining, and model serving of customized wake window predictions for young children
receiving inputs from a user taken by a app input device having a screen;
receiving nap times and wake times;
inputting the series of nap times and wake times into a data storage medium that is pre trained to analyze various aspects of the data, thereby extracting a plurality of features from the series of inputted data to train and analyze a machine learning model to generate internal prediction of optimal sleep window predictions based on the value of recent history.

2. The method of claim 1, wherein the machine learning model is pre trained by:
receiving a individualized dataset of wake and sleep windows;
receiving a ground truth wake and sleep windows determined by user input;
parsing each individualized features of into a series of dataset wake and sleep windows
Inputting the extracted features and the ground truth to the machine learning model, LightGBM, using industry standard techniques.

3. The method of claim 1, wherein the system receives a individualized dataset of wake and sleep windows
inputting such data into the data storage medium and
and a custom screening system designed by sleep experts is used to determine whether the individualized inputted dataset is within acceptable ranges and parsing and selecting the inputted data to determine appropriate values and features for predicted sleep windows.

4. The method of claim 1, wherein the system receives a individualized dataset of wake and sleep windows inputting such data into the data storage medium and and a custom screening system designed by sleep experts is used to determine whether the individualized inputted dataset is within acceptable ranges and if not in acceptable ranges parsing and selecting expert determined wake window standards to replace output generated from the inputted data to determine appropriate values and features for predicted sleep windows.

5. The method of claim 1, wherein the system is able to analyze various features of sleep and wake windows based on timing, trends, durations, and wake times.

6. The method of claim 1, further comprising the determination of at least one pattern between extracted features of sleep and wake windows; and associating each extracted feature with a labeled predicted sleep window based on each feature set.

7. The method of claim 6, wherein patterns are determined across the series of imputed datasets and normalized sleep and wake windows.

8. The method of claim 1, where certain inputted data features are given higher relevance with respect to inputted data features that are outside of accepted values.

9. The method of claim 1, where a rule to use the predictive models with current sleep values to present wake window predictions if the user has not been online for up to and including a certain minimum period of days.

10. The method of claim 1, where if the time the user has not logged in is greater than the optimized time period for inputted values, the value for the wake window determined by sleep experts is used as the prediction.
